(19) United States
(12) Patent Application Publication
(10) Pub. No.: US 2021/0077420 A1
(43) Pub. Date: Mar. 18, 2021

(54) Methods of Treatment of Gout

(71) Applicant: Mark Hooper, Oxfordshire (GB)

(72) Inventor: Mark Hooper, Oxfordshire (GB)

(21) Appl. No.: 16/772,609

(22) PCT Filed: Dec. 14, 2018
(86) PCT No.: PCT/GB2018/053628

§ 371 Date: Jun. 12, 2020

(30) Foreign Application Priority Data
Dec. 15, 2017 (GB) 1720992.5

(51) Int. Cl. A61K 31/05; A61K 9/107; A61K 31/724

(52) U.S. Cl. CPC A61K 31/05; A61K 45/06; A61K 31/724; A61K 9/1075

(57) ABSTRACT

This invention provides a solubility enhancer for monosodium urate for use in the treatment of gout. The solubility enhancer may be a pharmaceutically acceptable base, a solvent, a lipid, a surfactant, a pharmaceutically acceptable acid, a cyclodextrin, at least one paraben or a combination thereof.

The present invention relates to a medical use, and particularly to a treatment of gout.

Gout is a form of inflammatory arthritis. It is a significant global health problem with approximately 18 million patients affected in the US, EU and Japan combined. Gout is becoming increasingly common with the improvement of living standards and longer life expectancy, and is the most common form of inflammatory arthritis in men and post-menopausal women.

Gout is a disorder of purine metabolism. It is caused by the precipitation of uric acid (shown below) in the form of monosodium urate (MSU) monohydrate crystals into and around the joints of a patient. These MSU crystals cause an inflammatory response leading to the patient feeling pain.

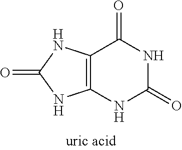 [SMILES: O=c1[nH]c(=O)c2[nH]c(=O)[nH]c2[nH]1]

The MSU crystals are commonly present in the synovial fluid of the affected joints of the patient or within the surrounding tissues, such as the synovial membrane or cartilage. The precipitation of these MSU crystals forms deposits in the joints of the patient known as “tophi”.

A preliminary stage of gout is asymptomatic hyperuricemia (i.e. elevated levels of uric acid in the blood). It is believed that the elevated level of uric acid in the blood causes an increased risk of gout, but the precise relationship is unknown. Many patients with asymptomatic hyperuricemia do not suffer from gout attacks.

Symptoms of gout include a sudden and intense pain around the affected joint, as well as swelling and erythema (redness). This pain normally occurs for 1 to 3 days, and often occurs during the night. The joint at the base of the big toe is the most common site for an acute gout attack. Other joints that can be affected include the ankles, knees, wrists, fingers, and elbows.

The initial phase of infrequent joint pain and gout attacks is known as acute gout. However, gout can develop into a chronic disease if left untreated. Intercritical gout occurs after the acute symptoms have been resolved, and low-grade inflammation may remain within the joint, causing unnoticed damage. During this stage, elevated uric acid levels in the blood drives precipitation of MSU crystals into the patient's affected joint (or joints) causing tophi development and erosive changes to the bone.

Chronic gout presents as persistent joint pain with repeated acute gout attacks, complicated by tophi formation.

At present, there are only limited treatment options for gout with no treatment offering a rapid treatment for the cause of gout. Typically, the initial aim of present treatments is to settle the symptoms of an acute gout attack by preventing inflammation. This is generally achieved using non-steroidal anti-inflammatory drugs (NSAIDs), colchicine or glucocorticosteroids. However, these treatments do not address the underlying cause of gout—crystallisation of MSU into the joints.

The most common long-term treatment for a patient suffering from gout is to focus on reducing the uric acid levels in the blood. This can be achieved by changing the patient's diet so that a lower supply of uric acid gets into the blood (by reducing purine intake). An alternate approach is through the use of drugs which suppress the production of uric acid or increase the excretion of uric acid.

Uric acid is formed in the body by the metabolism of purines, in particular the metabolism of xanthine by xanthine oxidases, and therefore any inhibitor of xanthine oxidase would eventually reduce uric acid levels in the blood. The idea behind these methods of treatment is to reduce blood uric acid to address crystal formation in the joints. However, it has been observed that even after reducing blood uric acid levels to acceptable or lower levels (such as about 6 mg per decilitre), it can take between two to three years for the MSU crystals to be removed (E. Pascual et al., Annal of Rheumatic Disease, 2007, 66, 2056-2058). Therefore, patients afflicted with gout often suffer significant pain and discomfort and further acute gout attacks whilst treatment is taking place.

There is therefore a need in the art for a more effective treatment of gout, in particular one that is simple to administer, effective at reducing symptoms, has low toxicity, and is less expensive than treatments that are currently available.

Accordingly, the present invention provides a solubility enhancer for monosodium urate for use in the treatment of grout. The invention also provides a solubility enhancer for monosodium urate for use in the treatment of gout by dissolving MSU crystals.

It has surprisingly been found that the use of a solubility enhancer of the present invention in the treatment of gout increases the solubility of MSU crystals and the rate at which MSU crystals dissolve in-vivo. In turn, this increased rate of dissolution of MSU crystals alleviates the suffering of a patient with gout and reduces the patient's risk of further acute gout attacks.

The solubility enhancer may be selected from at least one pharmaceutically acceptable base, at least one solvent, at least one lipid component, at least one surfactant, at least one pharmaceutically acceptable acid, at least one cyclodextrin, at least one paraben or a combination thereof. These “generally recognised as safe” excipients (as recognised by the FDA) increase the localised solubility of MSU crystals in the patient, providing an effective treatment of gout.

In one embodiment, the solubility enhancer is a pharmaceutically acceptable base and is selected from a metal carbonate, a metal hydroxide, a primary amine, a secondary amine, a tertiary amine, an aromatic amine, or a combination thereof.

In another embodiment, the solubility enhancer is a solvent and is selected from an alcohol, a diol, a polyol, an aryl or hereteroaryl alcohol, an arylalkyl or heteroarylalkyl alcohol, an ether, a polyether, a lactam, an amide, an alkyl sulfoxide, a ketone, an aldehyde, a nitrile, an ester, an isocyanide, or a combination thereof.

In another embodiment, the solubility enhancer is a lipid and is selected from C₄-C₂₈ carboxylic acids, C₁₁-C₂₈ alcohols, C₁-C₂₅ alkyl C₁-C₂₈ alkanoates, C₆-C₁₂ monoglycerides, C₆-C₁₂ diglycerides, C₆-C₁₂ triglycerides, C₁-C₂₈ alkyl N,N-disubstituted C₁-C₆ amino C₁-C₂₈ alkanoates, or a combination thereof.

In another embodiment, the solubility enhancer is a surfactant selected from a sorbitan ester, an ethoxylated sorbitan ester, a sorbitol ester, an ethoxylated sorbitol ester, a polyoxyethylated castor oil, a polyethoyxlated C₁₁-C₂₈ alcohol, a polyethoxylated C₄-C₂₈ carboxylic acid ester, a polyoxyethylene-polyoxypropylene block copolymer, or a combination thereof.

In another embodiment, the solubility enhancer is a pharmaceutically acceptable acid and is selected from C₁-C₇ carboxylic acids, C₂-C₁₀ dicarboxylic acids, C₁-C₅ alpha hydroxy acids, C₁-C₅ beta hydroxy acids, C₁-C₅ gamma hydroxy acids, sulfonic acids, or a combination thereof.

In another embodiment, the solubility enhancer is a cyclodextrin. Preferably, the cyclodextrin is a cyclodextrin having 6-8 glucopyranoside units. More preferably, the cyclodextrin is selected from alpha-cyclodextrin, hydroxypropyl-beta-cyclodextrin, or sulfobutylether-beta-cyclodextrin.

In another embodiment, the solubility enhancer is a paraben and the paraben is a C₁-C₂₀ alkyl paraben.

In another embodiment, the solubility enhancer is selected from sorbitan monooleate, fumaric acid, PEG-8 caprylic caprylo dilglyceride (sold as Labrasol®), dimethylformamide, tetraethylene glycol, N-methylpyrrolidone, isopropyl myristate, dimethylacetamide, geranyl acetate, PEG 200, PEG 300, polyoxyethylene (20) sorbitan monooleate, glucopon, benzyl alcohol, 4-hydroxybenzyl alcohol, triacetin, PEG-35 castor oil (sold as Cremophor® EL), oleic acid, PEG-40 hydrogenated castor oil (sold as Cremophor® RH40 and Kolliphor® RH40), lecithin (sold as Phosal® PG50), benzoic acid, 4-hydroxybenzoic acid, methyl paraben, propyl paraben, salicylic acid or a combination thereof.

In another embodiment, the solubility enhancer is PEG-40 hydrogenated castor oil, 2-(2-ethoxyethoxy)ethanol, or a combination thereof.

In another embodiment, the use comprises administering the solubility enhancer by injection to the affected area. This approach provides the advantage of delivering the solubility enhancer directly to the affected area. This allows the solubility enhancer to have high bioavailability and allows for rapid action of the solubility enhancer. The present invention therefore also provides for a syringe containing the present solubility enhancer.

In another embodiment, the use comprises administering the solubility enhancer transdermally to the affected area. This approach provides the advantage of administering the solubility enhancer directly to the affected area, whilst reducing the risk of systemic side-effects. When administered transdermally, the solubility enhancer can be administered in combination with a skin permeation enhancer. This provides the advantage of increasing the bioavailability of the transdermally administered solubility enhancer. The present invention therefore also provides for a transdermal patch containing the present solubility enhancer. Transdermal patches and their manufacture are generally known in the art, see for example EP 1 047 409. A transdermal patch may comprise: a layer remote from the skin, termed “backing layer”; a layer containing the solubility enhancer of the present invention, termed a “reservoir layer”; a layer facing the skin comprising a silicon polymer and a tackifier, termed “adhesive layer”; and a solubility enhancer impermeable layer, such as siliconized PET, siliconized polypropylene, siliconized polyethylene, fluoropolymer coated PET, fluoropolymer coated polypropylene, fluoropolymer coated polyethylene, which is removed from the patch prior to application.

In another embodiment, the use comprises administering the solubility enhancer in combination with at least one non-steroidal anti-inflammatory drug, at least one xanthine oxidase inhibitor, colchicine, at least one glucocorticosteroid, or a combination thereof. This embodiment may provide the advantage of improving the rate of dissolution of the MSU crystals and reducing the pain felt by the patient suffering from gout. The present invention therefore also provides for a syringe containing the present solubility enhancer and at least one non-steroidal anti-inflammatory drug. The present invention also provides for a transdermal patch containing the present solubility enhancer and at least one non-steroidal anti-inflammatory drug.

In another embodiment, the use comprises administering the solubility enhancer in combination with ultrasound therapy, heat therapy, and/or a change in diet to reduce uric acid levels in the patient. This embodiment may provide the advantage of improving the rate of dissolution of the MSU crystals.

The present invention also provides a method for treating gout comprising the step of administering an effective amount of the solubility enhancer of the present invention. It also provides for the use of the solubility enhancer of the present invention for the preparation of a medicament for the treatment of gout. It also provides for the use of the solubility enhancer of the present invention for the preparation of a medicament for the treatment of gout by dissolving MSU crystals.

The present invention will now be described with reference to the accompanying drawings, in which

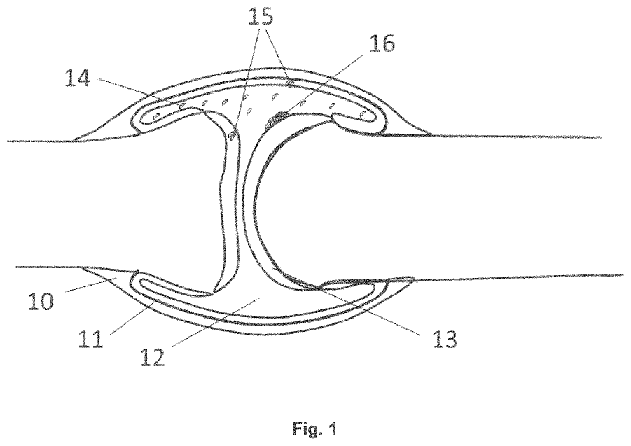

FIG. 1 shows a typical gout affected joint of a patient suffering from gout.

This invention provides a solubility enhancer for monosodium urate for use in the treatment of gout. The solubility enhancer may be a pharmaceutically acceptable base, a solvent, a lipid, a surfactant, a pharmaceutically acceptable acid, or a combination thereof.

The treatment promotes dissolution of the gout-causing monosodium urate crystals into the synovial fluid, providing a fast and effective gout treatment.

A typical gout-affected joint is shown in FIG. 1. The joint has an articular capsule 10, a synovial membrane 11, a cavity containing synovial fluid 12 and articular cartilage 13. As can be seen in the FIGURE, MSU crystals 14 form within the synovial fluid of the affected joint. These can be absorbed into the articular capsule or synovial membrane 15. If uric acid levels in the blood are high for prolonged periods then deposits of MSU crystals 16 may form in the joint.

It has been found that the solubility enhancer is able to increase the localised solubility of MSU crystals in the body (in particular, in the synovial fluid). This helps rapidly dissolve the MSU crystals, allowing for an effective treatment of gout. This treatment of gout effectively removes the MSU crystals from the afflicted joints in a timely manner.

The MSU is typically MSU monohydrate.

The solubility enhancing effect can be measured using a simple test:

a sample of 5 mg of MSU crystals are suspended in 4.75 mL of a phosphate buffered saline (made up of water with 0.14 mol/L NaCl and 0.01 N phosphate buffer ata pH of 7.4); 0.25 mL of solubility enhancer is added;
the sample is mixed for 16 hours;
an aliquot of the supernatant is taken, filtered and the concentration of MSU determined in solution;
the concentration is compared to a control sample without the solubility enhancer;
a positive result is found where more MSU is in solution than for the control.

The solubility enhancer of the present invention improves the solubility of monosodium urate. Typically, the solubility enhancer of the present invention improves the solubility by at least 5%. Preferably, the solubility enhancer of the present invention improves the solubility by at least 10%, more preferably by at least 20%, and most preferably by at least 30%. The increase is based on a comparison with control (i.e. the phosphate buffered saline described hereinabove without the solubility enhancer present).

The solubility enhancer may be selected from at least one pharmaceutically acceptable base, at least one solvent, at least one lipid component, at least one surfactant, at least one pharmaceutically acceptable acid, at least one cyclodextrin, at least one paraben or combinations thereof. Preferably, the solubility enhance is selected from selected from at least one pharmaceutically acceptable base, at least one solvent, at least one lipid component, at least one surfactant, at least one pharmaceutically acceptable acid, at least one cyclodextrin, or combinations thereof.

In one embodiment, the solubility enhancer is a pharmaceutically acceptable base and is selected from a metal carbonate, a metal hydroxide, ammonia, a primary amine, a secondary amine, a tertiary amine, a diamine, a nitrogen containing heteroaryl, a triamine, or a combination thereof.

Preferably, the pharmaceutically acceptable base is a metal carbonate, e.g. a group 1, group 2, group 3 or group 12 metal carbonate. More preferably the metal carbonate is selected from sodium carbonate, potassium carbonate, magnesium carbonate, calcium carbonate, aluminium carbonate or zinc carbonate.

Preferably, the pharmaceutically acceptable base is a metal hydroxide or a combination thereof. Preferably, the metal hydroxide is selected from a group 1, a group 2, a group 3, or a group 12 metal hydroxide. Even more preferably, the metal hydroxide is selected from sodium hydroxide, potassium hydroxide, magnesium hydroxide, calcium hydroxide, aluminium hydroxide or zinc hydroxide.

Alternatively, the pharmaceutically acceptable base may be a primary amine. Preferably, the primary amine is selected from a primary C₁-C₈ alkyl amine (such as ethylamine, tert-butylamine), lysine or tris(hydroxymethyl)aminomethane.

The pharmaceutically acceptable base may also be a secondary amine. Preferably, the secondary amine is selected from a di-C₁-C₈ alkyl substituted amine (such as dimethylamine and diethylamine) or meglumine.

The pharmaceutically acceptable base may also be a tertiary amine. Preferably, the tertiary amine is selected from a tri-C₁-C₈ alkyl substituted amine (such as trimethylamine and triethylamine) or procaine.

The pharmaceutically acceptable base may also be a diamine. Preferably, the diamine is selected from a diamine substituted C₁-C₈ alkyl (such as 1,2-diaminopropane and ethylene diamine) or benzathine.

The pharmaceutically acceptable base may also be a nitrogen-containing heteroaryl. Preferably, the nitrogen-containing heteroaryl is a 4-8 membered heteroaryl. More preferably, the nitrogen containing heteroaryl is pyridine.

The pharmaceutically acceptable base may also be a triamine. Preferably, the triamine is selected from a triamine substituted C₁-C₈ alkyl (such as diethylenetriamine).

As used herein, the term Cₙ-alkyl indicates an alkyl chain containing n carbon atoms. The alkyl chain may be branched or straight chain and may be mono-unsaturated, di-unsaturated, or polyunsaturated. The term Cₙ-aryl indicates an aryl ring containing n carbon atoms. For example C₆-aryl may indicate phenyl. The term Cₙ heteroaryl indicates an aryl ring containing n carbon atoms and up to 3 instances of a heteroatom independently selected from N, O or S. For example a C₅ heteroaryl may indicate pyridine.

In another embodiment, the solubility enhancer is a solvent and is selected from an alcohol, a diol, a polyol, an aryl alcohol, a hereteroaryl alcohol, an arylalkyl alcohol, a heteroarylalkyl alcohol, an ether, a polyether, a lactam, an amide, an alkyl sulfoxide, a ketone, an aldehyde, a nitrile, an ester, an isocyanide, a cyclodextrin, or a combination thereof.

The solvent may be an alcohol. Preferably, the alcohol is selected from C₁-C₁₀ alcohols. More preferably, the alcohol is selected from methanol, ethanol, propanol, butanol, menthol, or pentanol.

The solvent may be a diol. Preferably, the diol is selected from C₁-C₁₀ diols. More preferably, the diol is selected from monoethylene glycol, propylene glycol, 1,3-propanediol, 1,2-butanediol, 1,3-butanediol, 1,4-butanediol or 1,5-pentanediol.

The solvent may be a polyol. Preferably, the polyol is selected from C₁-C₁₀ polyols. More preferably, the polyol is selected from glycerol.

The solvent may be an aryl alcohol. Preferably, the aryl alcohol is selected from C₄-C₈ aryl alcohols or hydroxy substituted benzyl alcohols. More preferably, the aryl alcohol is 2-hydroxybenzyl alcohol, 3-hydroxybenzyl alcohol, 4-hydroxybenzyl alcohol, benzyl alcohol or d-alpha-tocopherol.

The solvent may be a heteroaryl alcohol. Preferably, the hereteroaryl alcohol is a C₄-C₈ heteroaryl alcohol.

The solvent may be an arylalkyl alcohol. Preferably, the arylalkyl alcohol is selected from a C₅-C₁₀ aryl C₄-C₈ alkyl alcohol.

The solvent may be a heteroarylalkyl alcohol. Preferably, the heteroarylalkyl alcohol is selected from a C₄-C₁₀ heteroaryl C₄-C₈ alkyl alcohol.

The solvent may be an ether. Preferably, the ether is a C₂-C₂₀ ether. More preferably, the ether is selected from dimethoxyethane, 1,4-dioxane or tetrahydrofuran (THF).

The solvent may be a polyether. Preferably, the polyether is a polyoxyethylene, a polyoxypropylene-polyoxyethylene copolymer, or a combination therefore. More preferably, the polyether is selected from PEG 200, PEG 300, PEG 400 tetraethylene glycol, poloxamer 188 (known as Pluronic® F-68) or poloxamer 407 (known as Pluronic F127), or poloxamer 182 (known as Pluronic L62).

The solvent may be a lactam. Preferably, the lactam is a C₃-C₇ lactam. More preferably, the lactam is N-methyl-2-pyrrolidine (NMP).

The solvent may be an amide. Preferably, the amide is a C₁-C₁₀ amide. More preferably the C₁-C₁₀ amide is selected from dimethylacetamide (DMA) and dimethylformamide (DMF).

The solvent may be an alkyl sulfoxide. Preferably, the alkyl sulfoxide is selected from a C₂-C₁₀ alkyl sulfoxide. More preferably, the C₂-C₁₀ alkyl sulfoxide is dimethylsulfoxide (DMSO).

The solvent may be a ketone. Preferably, the ketone is selected from a C₃-C₁₀ ketone. More preferably, the ketone is acetone or menthone.

The solvent may be an aldehyde. Preferably, the aldehyde is a C₁-C₁₀ aldehyde. More preferably, the aldehyde is acetaldehyde.

The solvent may be a nitrile. Preferably, the nitrile is a C₂-C₁₀ nitrile. More preferably, the nitrile is acetonitrile.

The solvent may be an ester. Preferably, the ester is a C₂-C₈ ester. More preferably, the ester is ethyl acetate, ethyl oleate or triacetin.

The solvent may be an isocyanide. Preferably, the isocyanide is selected from a C₂-C₁₀ isocyanide. More preferably, the isocyanide is selected from methyl isocyanide.

In another embodiment, the solubility enhancer is a lipid and is selected from a C₄-C₂₈ carboxylic acid, a C₁₁-C₂₈ alcohol, a C₁-C₂₈ alkyl C₁-C₂₈ alkanoate, a C₆-C₁₂ monoglyceride, a C₆-C₁₂ diglyceride, a C₆-C₁₂ triglyceride, a C₁-C₂₈ alkyl N,N-di-C₁-C₆-substituted amino C₁-C₂₈ alkanoate, C₁₀-C₃₀ alkanes, a phospholipid, or a combination thereof.

The lipid may be a C₄-C₂₈ carboxylic acid. More preferably, the lipid is a C₁₀-C₂₅ carboxylic acid. Preferably, the C₄-C₂₈ carboxylic acid is an omega-3, an omega-6 or an omega-9 fatty acid. Preferably, the C₄-C₂₈ carboxylic acid is selected from capric acid, oleic acid, linoleic acid, linolenic acid, decanoic acid, undecanoic acid, lauric acid, tridecanoic acid, myristic acid, pentadecanoic acid, palmitic acid, stearic acid, ethyloctadecanoic acid, linelaidic acid, neodecanoic acid, pelargonic acid, vaccenic acid, capric acid (decanoic acid), caproic acid (hexanoic acid), caprylic acid (octanoic acid), ricinoleic acid, undecylenic acid, benzoic acid or a hydroxy-substituted benzoic acid (e.g. 4-hydroxy benzoic acid, 3-hydroxy benzoic acid, 2-hydroxy benzoic acid). Preferably, the lipid is a combination of C₄-C₂₈ carboxylic acids such as those found in castor oil, cottonseed oil, olive oil, peanut oil, peppermint oil, safflower oil, sesame oil, soy fatty acids, soybean oil and hydrogenated soybean oils.

The lipid may be a C₁₁-C₂₈ alcohol. Preferably, the C₁₁-C₂₈ alcohol is selected from decanol, lauryl alcohol, linolenyl alcohol, nerolidol, 1-nonanol, n-octanol or oleyl alcohol.

The lipid may be a C₄-C₂₈ alkyl C₄-C₂₈ alkanoate. Preferably, the C₄-C₂₈ alkyl C₄-C₂₈ alkanoate is a C₆-C₂₅ alkyl C₆-C₂₅ alkanoate, more preferably a C₅-C₂₀ alkyl C₈-C₂₀ alkanoate. Preferably, the C₄-C₂₈ alkyl C₄-C₂₈ alkanoate is selected from isopropyl isostearate, isopropyl linoleate, isopropyl myristate, isopropyl palmitate, methyl acetate, methyl caprate, methyl laurate, methyl proprionate, methyl valerate, octyl acetate, oleyl oleate, ethyl acetate, ethyl propionate, geranyl acetate, butyl acetate, cetyl laurate, or 1-monocaproyl glycerol.

The lipid may be a C₆-C₁₂ alkyl monoglyceride. Preferably, the lipid component is a di-C₆-C₁₂ alkyl glyceride. Preferably, the lipid component is a tri-C₆-C₁₂ alkyl glyceride.

The lipid may be a C₁-C₂₈ alkyl N,N-di-C₁-C₆ alkyl substituted amino C₁-C₂₈ alkanoate. More preferably, the lipid component is a C₅-C₁₅ alkyl N,N-di-C₁-C₈ alkyl substituted amino C₁-C₁₀alkanoate. Preferably, the C₁-C₂₈ alkyl N,N-di-C₁-C₆ alkyl substituted amino C₁-C₂₈ alkanoate is selected from decyl N,N-dimethylamino acetate, decyl N,N-dimethylamino isopropionate, dodecyl N,N-dimethylamino acetate, dodecyl N,N-dimethylamino isopropionate, dodecyl N,N-dimethylamino butyrate, dodecyl 2-(dimethylamino)propionate, tetradecyl N,N-dimethylamino acetate or octyl N,N-dimethylamino acetate.

The lipid may be a phospholipid. Preferably, the phospholipid is selected from distearoylphosphatidylglycerol (also known as DSPG), L-alpha-dimyristoylphosphatidylcholine (DMPC), L-alpha-dimyristoylphosphatidylglycerol or 1-oleoyl-2-palmitoyl-phosphatidylcholine.

The lipid may be a miscellaneous lipid selected from diethyl sebacate, diethyl succinate, diisopropyl sebacate, ethylaceto acetate, glycerol monoethers, glycerol monolaurate, glycerol monooleate, glycerol monolinoleate, benzyl nicotinic ester, n-pentyl N-acetylprolinate, sucrose monooleate, or sucrose monolaurate.

In another embodiment, the solubility enhancer is a surfactant selected from a sorbitan ester, a sorbitol ester, an ethoxylated sorbitan ester, an ethoxylated sorbitol ester, a polyoxyl castor oil, an ethoxylated glycol alkyl ether, a polyoxyalkylene ester of a C₄-C₂₈ carboxylic acid, a sodium C₄-C₂₈ alkanoate, or a combination thereof.

The surfactant may be a sorbitan ester. Preferably, the sorbitan ester is a sorbitan mono-C₄-C₂₈ alkyl ester. More preferably, the sorbitan mono-C₄-C₂₈ alkyl ester is selected from sorbitan monolaurate (Span 20), sorbitan monooleate (Span 80), and sorbitan monopalmitate (Span 40). Preferably, the sorbitan ester is a sorbitan di-C₄-C₂₈ alkyl ester. More preferably, the sorbitan di-C₄-C₂₈ alkyl ester is selected from sorbitan dilautate, sorbitan dioleate. Preferably, the sorbitan ester is a sorbitan tri-C₄-C₂₈ alkyl ester. Preferably, the sorbitan tri-C₄-C₂₈ alkyl ester is selected from sorbitan trilaurate, sorbitan trioleate or sorbitan tristearate (Span 65).

The surfactant may be a sorbitol ester. Preferably, the sorbitol ester is a C₄-C₂₈ alkyl sorbitol ester.

The surfactant may be an ethoxylated sorbitan ester. Preferably the ethoxylated sorbitan ester is selected from polyoxyethylene (20) sorbitan monolaurate (Tween 20), polyoxyethylene (20) sorbitan monooleate (Tween 80), polyoxyethylene (20) sorbitan monopalmitate (Tween 40), polyoxyethylene (20) sorbitan monostearate (60), and polyoxyethylene (20) sorbitan trioleate (Tween 85).

The surfactant may be an alkoxylated castor oil. Preferably, the alkoxylated castor oil is selected from polyoxyl 35 castor oil (known as Cremophor® EL), polyoxyl 40 hydrogenated castor oil (Cremophor® RH40/Kolliphor® RH40), or polyoxyl 60 hydrogenated castor oil (Cremophor® RH60).

The surfactant may be a C₄-C₂₈ carboxylic acid polyoxyalkylene ester. Preferably, the C₄-C₂₈ carboxylic acid polyoxyalkylene ester is selected from PEG 300 oleic glyceride (also known as Labrafil® M-1944CS), PEG 300 linoleic glycerides (also known as Labrafil® M-2125CS), PEG 400 caprylic/capric glycerides (also known as Labrasol®), PEG 400 monostearate, PEG 1750 monostearate, lauroyl polyoxyl 32 glyceride (also known as Gellucire 44/14), stearoyl polyoxyl-32 glyceride (also known as Gellucire 50/13), PEG 300 caprylic/capric glyceride (also known as Softigen 767), polyethylene glycol (15)-hydroxystearate (known as Solutol® HS 15), or propylene Glycol monocaprylate (also known as Capmul PG-8 NF).

The surfactant may be a polyoxyethylene ether of a C₄-C₂₈ alcohol. Preferably, C₄-C₂₈ alcohol is substituted with between 2 and 100 oxyethylene units. More preferably, the polyoxyethylene ether of a C₄-C₂₈ alcohol is selected from polyoxyethylene (4) lauryl ether (Brij 30), ethoxylated dodecyl alcohol (Brij 36T), polyoxyethylene lauryl ether (Brij 35), polyoxyethylene (2) cetyl ether (Brij 52), polyoxyethylene (10) cetyl ether (Brij 56), polyoxyethylene (2) hexadecyl ether (Brij 58), polyoxyethylene (2) stearyl ether (Brij 72), polyoxyethylene 10 stearyl ether (Brij 76), polyoxyethylene (20) stearyl ether (Brij 78), polyoxyethylene (2) oleyl ether (Brij 92), polyoxyethylene (10) oleyl ether (Brij 96), or polyoxyethylene (2) oleyl ether (Brij-98).

The surfactant may be a sodium C₄-C₂₈ alkanoate. Preferably, the sodium C₄-C₂₈ alkanoate is selected from sodium laurate, or sodium oleate.

The surfactant may be a miscellaneous surfactant selected from d-alpha-tocopherol polyethylene glycol 1000 succinate (TPGS), cetyl trimethyl ammonium bromide, hydroxypolyethoxydodecane, lauroyl sarcsine, nonooxynol, octoxynol, phenylsulfonate, olyoxyethylene (8) nonyl phenol (known as Synperonic® NP), or 4-octylphenol polyethoxylate (known as Triton X-100).

In another embodiment, the solubility enhancer is a pharmaceutically acceptable acid and is selected from a C₁-C₇ carboxylic acid, a C₂-C₁₀ dicarboxylic acid, a C₁-C₅ hydroxy acid, a sulfonic acid, or a combination thereof.

Preferably, the pharmaceutically acceptable acid is a C₁-C₇ carboxylic acid. Preferably, the pharmaceutically acceptable acid is a C₁-C₃ carboxylic acid. Even more preferably, the C₁-C₃ carboxylic acid is formic acid, acetic acid, or propionic acid.

The pharmaceutically acceptable acid may be a C₂-C₁₀ dicarboxylic acid. Preferably, the C₂-C₁₀ dicarboxylic acid is selected from oxalic acid, malonic acid, sebacic acid, succinic acid, adipic acid, fumaric acid or maleic acid.

The pharmaceutically acceptable acid may be a C₁-C₅ hydroxy acid.

The pharmaceutically acceptable acid may be a sulfonic acid. Preferably, the sulfonic acid is selected from 2-hydroxyethanesulfonic acid, benzenesulfonic acid, camphor-10-sulfonic acid (+), ethane-1,2-disulfonic acid, ethanesulfonic acid, methanesulfonic acid, naphthalene-1,5-disulfonic acid, naphthalene-2-sulfonic acid, or p-toluenesulfonic acid.

The pharmaceutically acceptable acid may be a miscellaneous acid selected from 4-hydroxybenzoic acid, 1-hydroxy-2-naphthoic acid, 2,2-dichloroacetic acid, 2-oxoglutaric acid, 4-acetamidobenzoic acid, 4-aminosalicylic acid, ascorbic acid (L), aspartic acid (L), benzoic acid, camphoric acid (+), carbonic acid, cinnamic acid, citric acid, cyclamic acid, dodecylsulfuric acid, lactic acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, malic acid (−L), mandelic acid (DL), nicotinic acid, nitric acid, pamoic acid, phosphoric acid, pyroglutamic acid (−L), salicylic acid, sulfuric acid, tartaric acid (+L), or thiocyanic acid.

In another embodiment, the solubility enhancer is a cyclodextrin. Preferably, the cyclodextrin is a cyclodextrin having 6-8 glucopyranoside units. More preferably, the cyclodextrin is selected from alpha-cyclodextrin, hydroxypropyl-beta-cyclodextrin or sulfobutylether-beta-cyclodextrin.

In another embodiment, the solubility enhancer is a C₁-C₃₄ alkyl paraben (i.e. a C₁-C₃₄ alkyl ester of 4-hydroxybenzoic acid) or a combination thereof. Preferably the paraben is selected from methyl paraben, ethyl paraben, n-propyl paraben, isopropyl paraben, butyl paraben, isobutyl paraben, pentyl paraben, hexyl paraben, heptyl paraben, octyl paraben, nonyl paraben, decyl paraben, benzyl paraben, benzyl 4-hydroxybenzoate, salts thereof (for example, potassium salts), and/or combinations thereof. Even more preferably, the paraben is methyl paraben, ethyl paraben, propyl paraben, isopropyl paraben, butyl paraben.

In a preferred embodiment, the solubility enhancer is selected from sorbitan monooleate, fumaric acid, PEG-8 caprylic caprylo dilglyceride (sold as Labrasol®), dimethylformamide, tetraethylene glycol, N-methylpyrrolidone, isopropyl myristate, dimethylacetamide, geranyl acetate, PEG 200, PEG 300, polyoxyethylene (20) sorbitan monooleate, Glucopon, benzyl alcohol, triacetin, PEG-35 castor oil (sold as Cremophor® EL), oleic acid, PEG-40 hydrogenated castor oil (sold as Cremophor® RH40 and Kolliphor® 40), lecithin (sold as Phosal® PG50), benzoic acid, 4-hydroxybenzoic acid, methyl paraben, propyl paraben, salicylic acid or a combination thereof. More preferably, the solubility enhancer is selected from sorbitan monooleate, fumaric acid, PEG-8 caprylic caprylo dilglyceride (sold as Labrasol®), dimethylformamide, tetraethylene glycol, N-methylpyrrolidone, isopropyl myristate, dimethylacetamide, geranyl acetate, PEG 200, PEG 300, polyoxyethylene (20) sorbitan monooleate, Glucopon, benzyl alcohol, triacetin, PEG-35 castor oil (sold as Cremophor® EL), oleic acid, PEG-40 hydrogenated castor oil (sold as Cremophor® RH40 and Kolliphor® 40), lecithin (sold as Phosal® PG50), or a combination thereof.

Even more preferably, the solubility enhancer is PEG-40 hydrogenated castor oil, 2-(2-ethoxyethoxy)ethanol, oleic acid, benzyl alcohol, 4-hydroxy benzyl alcohol, benzoic acid, 4-hydroxybenzoic acid, methyl paraben, propyl paraben or a combination thereof. Even more preferably, the solubility enhancer is PEG-40 hydrogenated castor oil, 2-(2-ethoxyethoxy)ethanol, or a combination thereof.

Many of the solubility enhancers disclosed herein can exist in the form of salts, for examples alkali metal salts. All such salts are within the scope of this invention, and references to the solubility enhancers of the present invention include the salt forms, e.g. sodium salt, potassium salt, magnesium salt. The salts of the present invention can be synthesized from the parent solubility enhancer that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of the solubility enhancer with the appropriate base or acid in water or in an organic solvent, or in a mixture of the two; generally, nonaqueous media such as ether, ethyl acetate, ethanol, isopropanol, or acetonitrile are used. Examples of pharmaceutically acceptable salts are discussed in Berge et al., 1977, “Pharmaceutically Acceptable Salts,” J. Pharm. Sci., Vol. 66, pp. 1-19. Preferred salts of the present invention include lithium benzyloxide, lithium benzoate, lithium 4-hydroxybenzoate, sodium benzyloxide, sodium benzoate, sodium 4-hydroxybenzoate, potassium benzyloxide, potassium benzoate, potassium 4-hydroxybenzoate, calcium benzyloxide, calcium benzoate, calcium 4-hydroxybenzoate, magnesium benzyloxide, magnesium benzoate, and magnesium 4-hydroxybenzoate.

The amount of solubility enhancer administered will depend on the route of administration, the joint affected and the size of the patient. Typical amounts administered are 0.1-10 g per application. The solubility enhancer may be administered b.i.d. or q.d, or less frequently, such as once per week.

The solubility enhancer of the present invention may be administered parenterally. In one embodiment, the solubility enhancer is administered by injection to the affected area.

Standard formulation and manufacturing techniques can be used to produce a suitable stable, sterile vehicle for injection containing the solubility enhancer of the present invention. This could be administered directly into the areas of the body where MSU crystals are present, such as the synovial fluid around the joint. The advantage of this approach is that the solubilising formulation is delivered directly to the site of action. The disadvantage is that a medical professional would be needed to administer the injection(s).

In another embodiment, the solubility enhancer is administered transdermally to the affected area. Again, standard formulation and manufacturing techniques can be used to produce a suitable formulation. This approach provides the advantage of administering the solubility enhancer directly to the affected area, whilst reducing the risk of systemic side-effects.

When administered transdermally, the solubility enhancer may be administered in combination with a skin permeation enhancer (also known as skin penetration enhancers). These are components which are known to help deliver drugs and excipients through the skin. Examples of suitable skin penetration enhancers may be found in: “Skin Penetration Enhancers Cited in the Technical Literature”. D. W. Osbourne and J. J. Henke, Pharmaceutical Technology, November 1997; page 58; “Permeation Enhancers for Transdermal Drug Delivery”, V. R. Sinha and M. Pal Kaur; Drug Development and Industrial Pharmacy, 2000, 26(11), 1131-1140; and “Chemical Penetration Enhancers for Transdermal Drug Delivery Systems”, I. B. Pathan and C. M. Setty, Tropical Journal of Pharmaceutical Research, April 2009, 8(2), 173-179. Non-limiting examples include Transcutol® (also known as 2-(2-ethoxyethoxy)ethanol) and dodecyl 2-N,N-dimethylaminopropionate.

The solubility enhancer of the present invention may also be administered in the form of a cream, a gel for topical application. This could require the inclusion of thickening agents such as carbomer, hydroxypropylcellulose (HPC, sold as Klucel®), glycerol dibehenate (sold as Compritol® 888), glycerol monostearate (Gelot®), (sold as Sedefos®), stearic acid, hydroxyethylcellulose, propylene glycol alginate.

A further aspect of the present invention, involves administering the solubility enhancer in the form of a microemulsion. Microemulsions are thermodynamically stable systems of oil, water and a surfactant (and optionally a co-surfactant) with a droplet size in the range 1 to 100 nm, usually 10 to 50 nm. These systems have advantages such as thermodynamic stability, increased permeability and delivery of drugs transdermally, enhanced drug solubility, high biocompatibility and facile preparation. Owing to the very small droplet size, microemulsions have very low surface tension and a large interfacial area. Specific advantages for using a microemulsion in the preferred systems described in this patent include a simple stable homogeneous system to be topically applied or injected to the affect area. This allows for the largest bioavailability of the solubility enhancers to the gout affected region.

In one embodiment, the solubility enhancer of the present invention is administered in the form of a microemulsion composition comprising an oil, water, and a surfactant. In one embodiment, the solubility enhancer of the present invention is selected to be a lipid and is selected from the lipids described previously. In this embodiment, the solubility enhancer may form part of the oil component of the microemulsion. The other components of the microemulsion may be selected using standard formulation techniques available in the art.

In another embodiment, the solubility enhancer of the present invention is selected to be a surfactant and is selected from those described previously. In this embodiment, the solubility enhancer may form part of the surfactant or co-surfactant component of the microemulsion.

It is therefore apparent that the present invention provides for a pharmaceutical composition comprising the solubility enhancer of the present invention and one or more excipients (which are different to the solubility enhancer).

The solubility enhancer of the present invention may also be administered in combination with at least one non-steroidal anti-inflammatory drug (also known as NSAIDs), at least one xanthine oxidase inhibitor, colchicine, at least one glucocorticosteroid, or a combination thereof.

Examples of NSAIDs include aminoarylcarboxylic acid derivatives such as enfenamic acid, etofenamate, flufenamic acid, isonixin, meclofenamic acid, mefenamic acid, niflumic acid, talniflumate, terofenamate, and tolfenamic acid; arylacetic acid derivatives such as aceclofenac, acemetacin, alcofenac, amfenac, amtolmetin guacil, bromfenac, bufexamac, cinmetacin, clopirac, diclofenac sodium, diclofenac, other diclofenac salts, diclofenac diethylammonium, diclofenac potassium, etodolac, felbinac, fenclozic acid, fentiazac, glucametacin, ibufenac, indomethacin, isofezolac, isoxepac, lonazonlac, metiazinic acid, mofezolac, oxametacin, pirazolac, proglmetacin, sulindac, tiaramide, tolmetin, tropesin, zomepirac; arylbutyric acid derivatives such as bumadizon, butibufen, fenbufen, xenbucin; arylcarboxylic acids such as clidanac, ketorolac, tinoridine; arylpropionic acid derivatives such as alminoprofen, benoxaprofin, bermaprofen, bucloxic acid, carprofen, fenoprofen, flunoxaprofen, flurbiprofen, ibuprofen, salts of ibuprofen, ibuproxam, indoprofen, ketoprofen, loxoprofen, naproxen, oxoaprozin, piketoprofin, pirprofen, pranoprofen, protizinic acid, suprofen, tiaprofenic acid, ximoprofen, zaltoprofen; pyrazoles such as difenamizole, epiraozole; pyrazolones such as apazone, benzpiperylon, feprazone, mofebutazone, morazone, oxyphenbutazone, phenylbutazone, pipebuzone, propyphenazone, prostaglandins, ramifenazone, suxibuzone, thiazolinobutazone; salicylic acid derivatives such as acetaminosalol, aspirin, benorylate, bromosaligenin, calcium acetylsalicylate, diflusinal, etersalate, fendosal, gentisic acid, glycol salicylate, imidazole salicylate, lysine acetyl salicylate, mesalamine, morphline salicylate, 1-naphthyl salicylate, olsalazine, parsalmide, phenyl acetyl salicylate, phenyl salicylate, salacetamide, salicylamide o-acetic acid, salicylsulfuric acid, salsalate, sulfasalazine; thiazinecarboxamides such as ampiroxicam, droxicam, isoxicam, lomoxicam, piroxicam, tenoxicam; cyclooxygenase-II inhibitors (COX-II inhibitors) such as Celebrex (Celecoxib), Vioxx, Relafen, Lodine, Voltaren; and others such as epsilon-acetamidocaproic acid, s-adenosylmethionine, 3-amino-4-hyroxybutyric acid, amixetrine, bendazac, benzydamine, alpha-bisabolol, bucololome, difenpiramide, ditazol, emorfazone, fepredinol, guaiazulene, nabumatone, nimesulide, oxaceprol, paranyline, perisoxal, proquazone, tenidap, zilenton.

Examples of xanthine oxidase inhibitors include purine analogues such as allopurinol, oxypurinol and tisopurine; and others such as febuxostat, topiroxostat, and inositols.

Examples of glucocorticosteroids include 11-dehydrocorticosterone, 11-deoxycorticosterone, 11-deoxycortisol, 11-ketoprogesterone, 11β-hydroxypregnenolone, 11β-hydroxyprogesterone, 11β,17α,21-trihydroxypregnenolone, 17α,21-dihydroxypregnenolone, 17α-hydroxypregnenolone, 17α-hydroxyprogesterone, 18-hydroxy-11-deoxycorticosterone, 18-hydroxycorticosterone, 18-hydroxyprogesterone, 21-deoxycortisol, 21-deoxycortisone, 21-hydroxypregnenolone, aldosterone, corticosterone, cortisol, cortisone, pregnenolone, progesterone, flugestone, fluorometholone, medrysone, prebediolone acetate, chloroprednisone, cloprednol, difluprednate, fludrocortisone, fluocinolone, fluperolone, fluprednisolone, loteprednol, methylprednisolone, prednicarbate, prednisolone, prednisone, tixocortol, triamcinolone, dexamethasone, alclometasone, beclometasone, betamethasone, clobetasol, clobetasone, clocortolone, desoximetasone, diflorasone, difluocortolone, fluclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol, amcinonide, budesonide, ciclesonide, deflazacort, desonide, formocortal, fluclorolone acetonide, fludroxycortide, flunisolide fluocinolone acetonide, fluocinonide, halcinonide, triamcinolone acetonide and cortivazol.

In another embodiment, the use comprises administering the solubility enhancer in combination with ultrasound therapy, heat therapy, a change in diet to reduce uric acid levels in the patient.

The use of ultrasound therapy from a non-contact distance via a liquid spray to reduce inflammation in inflammatory disorders (including gout) is mentioned in US 2009/0177123. This method is generally used for wound treatment only and is commercially available from Alliqua Biomedical Inc. as “MIST Therapy”. However, the ultrasound is used specifically to decrease the inflammatory response. There is no mention of using ultrasound to aid dissolution of MSU crystals in situ.

The present invention may use ultrasound at therapeutic levels applied externally in contact with the skin around the area affected by gout. This ultrasound aids the dissolution of the MSU crystals in a number of ways, such as providing some localised perturbation of the crystals which can help the flow of aqueous media around the crystals, helping dissolution. Another action of the ultrasound is to help break up the crystals in situ which can increase the rate of dissolution. There is also some localised gentle warming which can also aid dissolution.

The ultrasound may be supplied from a single-source device similar to those that are commercially available. Preferably, the ultrasound may be delivered from two or more sources as a confocal ultrasound device to focus more of the ultrasound energy in the specific target areas, i.e. where the MSU crystals are deposited (e.g. synovial fluid and tissue in and around a joint).

When used with a transdermally delivered solubility enhancer, there may be the added advantage of enhancement of percutaneous delivery of the solubilising system formulation via phonophoresis (PH). The therapeutic ultrasound can aid skin penetration for drugs and chemicals (as reported in “Physical enhancement of dermatologic drug delivery: Intophoresis and phonophoresis”, D. G. Kassan, A. M. Lynch and M. J. Stiller; J Am Acad Dermatol, 1996, 34, 657-66).

Typically, ultrasound at 1-3 MHz is used for therapeutic treatment. There are generally two different modes of administering the ultrasound therapy, either continuous or modulated. Continuous wave ultrasound uses an unmodulated beam with intensities often limited to 0.5-2.5 W/cm². This mode is typically responsible for a concomitant heating effect. Modulated ultrasound uses a modulated beam to deliver brief pauses on no power. This is generally associated with little or no heating effect. Either approach may be used to help increase the rate of dissolution of the MSU crystals and treating gout.

Heat therapy may also be used in combination with the solubility enhancer of the present invention. Heat therapy is often applied to obtain analgesia, decrease muscle spasm, increase collagen extensibility and accelerate metabolic processes. Two forms of heat therapy are generally available.

Superficial agents such as hot packs warm the skin and subcutaneous tissues or deep heating agents such as therapeutic ultrasound can produce a raise in temperature of 4-5° C. at depths of 8 cm. Either approach provides viable options for gently warming the environment in which the MSU crystals can dissolve within the body, and therefore can help increase the rate of dissolution of the MSU crystals and treating gout.

There are a full range of treatments available to help reduce the level of urate in the body fluids over the long term either through changes in diet (lowering supply of purines that are metabolised into uric acid), or that increase the rate of uric acid elimination from the body (such as Probenecid), or drug/enzymes that help break down uric acid (such as Rasburicase).

To aid the rate of removal of dissolved MSU, the perfusion of urate from the synovial fluids into the blood and out of the body needs to be encouraged. Small molecules, such as urate, pass easily and rapidly from synovial fluids into the blood, so measures to reduce the level of urate in the blood and encourage clearance of urate from the blood will aid clearance of urate from the synovial fluid and consequently from around the MSU crystals (as explained in “Synovial perfusion and synovial fluid solutes”, P. A Simkin, Annal of the Rheumatic Disease, 1995, 54, 424-428).

Measures to reduce blood MSU levels the in the short term include drinking water and reducing consumption of purine rich foods. Increasing the water intake of a patient has been shown to reduce the chances of gout attacks in patients (“Study on dehydration and gout”, Annual Meeting of the American College of Rheumatology, Tuhina Neogi, 2009, Philadelphia). Another option is to reduce the intake of purine rich foods such as organ meats (e.g. liver, kidneys, sweetbreads and brains) and general meats (such as bacon, beef, pork and lamb), oily fish (such as anchovies, sardines, herring and mackerel) and beer.

It is preferable to use this dilution approach in combination with administration of the solubility enhancer of the present invention.

The present invention will now be described with reference to the following non-limiting examples.

### EXAMPLES

### Example 1

### Solubility Enhancement of Monosodium Urate Crystals in Aqueous NaCl Solution

A NaCl solution was prepared by dissolving 8.2 g of NaCl in 1 L of deionised water. Approximately 8 mg of monosodium urate crystals was then suspended in 2.5 mL of the NaCl solution. A 2.5 mL amount of a solubility enhancer was then added to the suspension of monosodium urate crystals. In the case of the solubility enhancer being a mix of components, equal portions by volume of each component was added. This was then mixed for about 16 hours on a roller mixer.

A sample of the supernatant was then taken, filtered using standard techniques (e.g. a syringe filter), and analysed by high-performance liquid chromatography (HPLC) to determine the concentration uric acid/MSU present in solution. The results are shown below in Table 1.

TABLE 1
Solubility enhancement of MSU crystals in aqueous NaCl solution

| | Concentration | |
|---|---|---|
| | of urate | % vs |
| Solubility enhancer | (mg/mL) | control |
| Control (water only) | 0.043 | 100% |
| Propylene glycol | 0.049 | 114% |
| Tween 20 | 0.049 | 114% |
| Poloxamer | 0.052 | 121% |
| Solutol HS15 | 0.053 | 123% |
| Dimethyl sulfoxide | 0.054 | 126% |
| Miglyol 812 | 0.055 | 128% |
| Capmul PG-8 | 0.056 | 130% |
| PEG 400 | 0.057 | 133% |
| Span 85 | 0.057 | 133% |
| PEG 300/Tween 20 | 0.062 | 144% |
| Labrafil | 0.063 | 147% |
| Dimethyl acetamide/Tween20/ | 0.064 | 149% |
| PEG 300 | | |
| Glycerol | 0.064 | 149% |
| PEG 300/Glucopon | 0.064 | 149% |
| Sorbitan monooleate | 0.065 | 151% |
| Fumaric acid | 0.065 | 151% |
| Labrasol | 0.066 | 153% |
| Dimethylformamide | 0.069 | 160% |
| Lecithin | 0.069 | 160% |
| Tetraglycol | 0.080 | 186% |
| N-methylpyrrolidone | 0.086 | 200% |
| Isopropyl myristate | 0.087 | 202% |
| Dimethylacetamide | 0.088 | 205% |
| Geranyl acetate | 0.090 | 209% |
| PEG 300 | 0.095 | 221% |
| Tween 80 | 0.104 | 242% |
| Glucopon | 0.104 | 242% |
| Benzyl alcohol | 0.107 | 249% |
| Cremophor EL | 0.108 | 251% |
| Oleic acid/geranyl acetate | 0.109 | 253% |
| PEG 200 | 0.111 | 258% |
| Benzyl alcohol/Glucopon | 0.118 | 274% |
| Oleic acid | 0.123 | 286% |
| Cremophor RH40 | 0.131 | 305% |
| Phosal PG50 | 0.135 | 314% |
| Oleic acid/Glucopon | 0.146 | 340% |
| Oleic acid/Cremophor RH40 | 0.148 | 344% |

All samples were analysed using a HPLC system equipped with a variable wavelength UV detector and a reversed phase column (5 μm ODS2, 4.6 mm×150 mm, Waters Spherisorb). The mobile phase was 35 mM sodium acetate in water with pH adjusted to 5.0 using acetic acid. The flow rate was set at 1 mL/min and UV detection at a wavelength of 292 nm. Sample injection volume was 50.0 μL and all operations were carried out at 25° C. Standard solutions of MSU were made up in the range of 15-250 μg/mL and analysed as above. The peak area correlated linearly with the MSU concentration in the tested range of 15-250 μg/mL, with an average correlation coefficient of 0.993.

Although the present examples have been carried out at room temperature (25° C.) for convenience, the observed increases in solubility of MSU would correlate similarly to those that would be observed at 37° C. (as explained by Kippen et al., “Factors affecting urate solubility in vitro”, Annals of Rheumatic Diseases, 1974, 33, 313-317).

### Example 2A

### Solubility Enhancement of Monosodium Urate Crystals in Phosphate Buffered Saline

A phosphate buffered saline diluent was prepared by dissolving 8.2 g of NaCl and 0.68 g of KH₂PO₄ in 500 mL of deionised water in a 1 L flask. An NaOH solution was prepared by dissolving 0.399 g of NaOH in 100 mL of deionised water. 39.1 mL of the 0.1 M NaOH solution was then added to the 1 L flask and the volume was made up to 1 L with deionised water. The pH was adjusted to 7.4.

Approximately 5 mg of monosodium urate crystals was then suspended in 4.75 mL of the phosphate buffered saline diluent. A 0.25 mL amount of a solubility enhancer was then added to the suspension of monosodium urate crystals. This provided a 5% by volume solubility enhancer experiment, unless otherwise indicated in Table 2 below. Where a different percentage is indicated in Table 2, the amounts of the phosphate buffered saline diluent and the solubility enhancer were varied to provide a total volume of 5 mL with the specified % by volume of the solubility enhancer. In the case of the solubility enhancer being a mix of components, equal portions by volume of each component was added, unless indicated otherwise. This was then stirred for about 16 hours.

A sample of the supernatant was then taken, filtered using standard techniques (e.g. a syringe filter), and analysed by high-performance liquid chromatography (HPLC) to determine the concentration of uric acid/urate present in solution. The results are shown below in Table 2.

TABLE 2
Solubility enhancement of MSU crystals
in phosphate buffered saline solution

| | Concentration | |
|---|---|---|
| | of urate | % vs |
| Solubility enhancer | (mg/mL) | control |
| Control (Phosphate buffered saline | 0.040 | 100% |
| diluent only) | | |
| Kolliphor RH40 | 0.044 | 111% |
| Ethylene glycol | 0.045 | 112% |
| Linoleic acid | 0.046 | 115% |
| Glycerol | 0.046 | 114% |
| Propylene glycol | 0.046 | 114% |
| Transcutol/Propylene glycol | 0.047 | 117% |
| 3% PEG200/1% Transcutol/1% Labrasol | 0.047 | 117% |
| Methyl oleate | 0.048 | 119% |
| Transcutol/Glycerol | 0.049 | 121% |
| Polysorbate 20 | 0.049 | 123% |
| Transcutol/Ethylene glycol | 0.050 | 126% |
| 1% Transcutol | 0.051 | 128% |
| PEG 200 | 0.051 | 128% |
| 2.5% Transcutol | 0.053 | 131% |
| Transcutol/Triacetin | 0.053 | 132% |
| 1.7% Oleic acid + 1.7% RH40 + | 0.053 | 134% |
| 1.7% Transcutol | | |
| PEG 200/Transcutol | 0.053 | 133% |
| Transcutol/Labrasol | 0.054 | 134% |
| 5% Transcutol | 0.058 | 144% |
| Transcutol/Lauroglycol | 0.059 | 148% |
| 10% Transcutol | 0.061 | 153% |
| 1.7% Oleic acid + 1.7% Transcutol + | 0.063 | 157% |
| 1.7% Benzyl alcohol | | |
| 2.5% Formulation A* | 0.066 | 164% |
| Triacetin | 0.066 | 165% |
| Oleic acid/Kolliphor RH40 | 0.071 | 179% |
| Benzyl alcohol/Transcutol | 0.075 | 187% |
| Benzyl alcohol/PEG 200 | 0.097 | 241% |
| Formulation A | 0.103 | 257% |
| Benzyl alcohol | 0.105 | 263% |

*Formulation A comprised 10% oleic acid/26.7% Labrasol/8.3% Transcutol/5% Compritol/50% water.

As explained by Kippen et al., ibid, the above-described phosphate buffered saline diluent system is representative of the solubility of sodium urate/uric acid in the synovial fluid and plasma of a patient.

### Example 2B

### Further Examples of Solubility Enhancement of Monosodium Urate Crystals in Phosphate Buffered Saline

The ability of the following components to enhance the solubility of monosodium urate crystals in phosphate buffered saline enhancers was tested according to the procedure outlined in Example 2A. The results are shown below in Table 3.

TABLE 3
Solubility enhancement of MSU crystals
in phosphate buffered saline solution

| | Concentration | |
|---|---|---|
| | of urate | % vs |
| Solubility enhancer | (mg/mL) | control |
| Control (Phosphate buffered saline | 0.040 | 100% |
| diluent only) | | |
| 4% Triacetin | 0.044 | 111% |
| Triacetin | 0.045 | 113% |
| 2% Oleic acid + 2% Kolliphor RH40 | 0.046 | 115% |
| Propyl paraben | 0.046 | 115% |
| 1% Benzyl alcohol + 1% Ethylene glycol | 0.046 | 115% |
| 1% Transcutol + 2% Kolliphor RH40 + | 0.047 | 117% |
| 1% Benzyl alcohol | | |
| 1% Oleic acid | 0.047 | 118% |
| 1% Benzyl alcohol + 1% Span-85 | 0.048 | 119% |
| 2% Oleic acid | 0.048 | 119% |
| 1% Benzyl alcohol + 1% Propylene | 0.047 | 119% |
| glycol | | |
| 1% Benzyl alcohol + 1% PEG-200 | 0.048 | 120% |
| 1% Benzyl alcohol + 1% Transcutol | 0.049 | 121% |
| 1% Polysorbate 20 | 0.048 | 121% |
| 1% Benzyl alcohol + 1% Glycerol | 0.049 | 123% |
| 1% Benzyl alcohol + 1% Kolliphor | 0.053 | 132% |
| RH40 | | |
| 1% Methyl paraben | 0.053 | 133% |
| 2% Labrasol | 0.054 | 136% |
| 1% Propyl paraben | 0.055 | 137% |
| 1% Benzyl alcohol + 1% Triacetin | 0.056 | 140% |
| 1% Benzyl alcohol + 1% Kolliphor | 0.057 | 144% |
| RH40 + 1% Triacetin | | |
| Labrasol | 0.057 | 144% |
| 0.1% Benzoic acid | 0.059 | 147% |
| Polysorbate 20 | 0.059 | 148% |
| 0.3% Formulation B* | 0.059 | 148% |
| 0.33% Benzyl alcohol + 0.33% | 0.061 | 151% |
| Labrasol + 0.33% Polysorbate 20 | | |
| 1% 4-hydroxybenzyl alcohol | 0.060 | 151% |
| 0.5% Benzyl alcohol | 0.061 | 152% |
| 2% Polysorbate 20 | 0.061 | 152% |
| 1% Benzyl alcohol + 1% Labrasol | 0.061 | 152% |
| 0.16% Benzyl alcohol + 0.16% | 0.062 | 156% |
| Triacetin + 0.16% Polysorbate 20 | | |
| 5% Methyl paraben | 0.063 | 157% |
| 0.33% Benzyl alcohol + 0.33% | 0.064 | 159% |
| Triacetin + 0.33% Polysorbate 20 | | |
| 1% Benzyl alcohol + 1% Polysorbate- | 0.064 | 159% |
| 20 | | |
| 1% Benzoic acid | 0.063 | 159% |
| 0.05% 4-hydroxybenzoic acid | 0.064 | 160% |
| 0.5% 4-hydroxybenzoic acid + 1% | 0.064 | 160% |
| Polysorbate 20 | | |
| 0.5% 4-hydroxybenzoic acid + 1% | 0.065 | 163% |
| RH40 | | |
| 1% Labrasol | 0.065 | 164% |
| 1% Benzyl alcohol + 1% Labrasol + | 0.067 | 168% |
| 1% Triacetin | | |
| 2% Benzyl alcohol | 0.068 | 170% |
| 0.03% Benzyl alcohol + 0.03% | 0.069 | 172% |
| Triacetin + 0.03% Polysorbate 20 | | |
| 0.5% 4-hydroxybenzoic acid + 1% | 0.070 | 175% |
| Triacetin | | |
| 0.5% 4-hydroxybenzoic acid + 1% | 0.071 | 178% |
| Labrasol | | |
| 0.5% 4-hydroxybenzoic acid + 1% | 0.071 | 178% |
| Formulation B | | |
| 0.016% Benzyl alcohol + 0.016% | 0.073 | 183% |
| Labrasol + 0.016% Polysorbate 20 | | |
| 1% Benzyl alcohol & 1% Labrasol + | 0.075 | 188% |
| 1% Polysorbate 20 | | |
| Benzoic acid | 0.076 | 189% |
| 0.03% Benzyl alcohol + 0.03% | 0.076 | 190% |
| Labrasol + 0.03% Polysorbate 20 | | |
| 4-hydroxybenzyl alcohol | 0.076 | 190% |
| 0.1% 4-hydroxybenzoic acid | 0.077 | 193% |
| 3% Benzyl alcohol | 0.079 | 197% |
| 0.5% 4-hydroxybenzoic acid + 0.5% | 0.080 | 200% |
| Formulation B | | |
| 1% 4-hydroxybenzoic acid | 0.082 | 205% |
| 1% Benzyl alcohol + 1% Labrasol + | 0.083 | 208% |
| 1% RH40 | | |
| 4% Benzyl alcohol | 0.091 | 227% |
| 1% Formulation B | 0.093 | 233% |
| 0.5% 4-hydroxybenzoic acid & 1% | 0.094 | 234% |
| Benzyl alcohol | | |
| 1% Benzyl alcohol + 1% Triacetin + | 0.095 | 238% |
| 1% Polysorbate 20 | | |
| 0.5% 4-hydroxybenzoic acid + 1% | 0.095 | 238% |
| Labrasol + 1% Polysorbate 20 | | |
| 4-hydroxybenzoic acid | 0.097 | 241% |
| 1% Formulation A | 0.099 | 246% |
| 0.05% Benzyl alcohol + 0.05% | 0.106 | 265% |
| Formulation B | | |
| Formulation B | 0.110 | 274% |
| 0.1% Salicylic acid | 0.112 | 281% |
| 0.5% 4-hydroxybenzoic acid + 1% | 0.116 | 290% |
| Benzyl alcohol + 1% Polysorbate 20 | | |
| 3% Formulation A | 0.148 | 370% |
| 0.25% Benzyl alcohol + 0.25% | 0.156 | 390% |
| Formulation B | | |
| 0.5% Benzyl alcohol + 0.5% | 0.233 | 583% |
| Formulation B | | |

*Formulation B comprised 10% oleic acid/26.7% Labrasol/8.3% Transcutol/55% water.

Formulation A was as described in Example 2A.

As explained by Kippen et al., ibid, the above-described PBS diluent system is representative of the solubility of sodium urate/uric acid in the synovial fluid and plasma of a patient. This example therefore demonstrates the ability of the claimed solubility enhancers to treat gout.

### Example 3

### Solubility Enhancing Combinations

The ability of a combination of solubility enhancers of the present invention to increase the localised solubility of monosodium urate crystals when administered transdermally was tested using the following method.

The compositions comprised Component 1 (triacetin or benzyl alcohol or PEG-200 or glycerol or propylene glycol or Labrasol or Capryol or Lauroglycol or geranyl acetate), Kolliphor® RH40 and Transcutol (2-(2-ethoxyethoxy)ethanol) as indicated in Table 4 below.

Component 1, Kolliphor® RH40 and Transcutol were added to a vial in the required amounts. The ratios (by volume) of component 1:Kolliphor® RH40:Transcutol are given below in Table 4. These components were mixed with stirring at approximately 500 rpm for 30 minutes to form a solubility enhancing composition of the present invention.

A phosphate buffered saline diluent system was prepared to provide a representative imitation of the ionic mixture present in the blood and synovial fluid, similar to Example 2. An aqueous solution of 0.14 mol/L NaCl was prepared and a 0.01 N phosphate buffer. This solution had a pH of 7.4 and is referred to as “PBS” hereafter.

Approximately 5 mg of MSU crystals was suspended in 4.75 mL of diluent, and then 0.25 mL of the formulation was added. This was stirred for 16 hours at room temperature. A sample of the supernatant was taken, filtered through a syringe filter and analysed by HPLC to determine the concentration of uric acid/urate in solution. Results are shown below in Table 4.

TABLE 4
Solubility enhancement of MSU crystals
using various formulations

| Composition | Concentration | |
|---|---|---|
| (component 1:Kolliphor ® | of Urate | % vs |
| RH40:Transcutol) | (mg/mL) | control |
| Control (PBS solution only) | 0.040 | 100% |
| Triacetin 30%:20%:50% | 0.057 | 142% |
| Benzyl alcohol 30%:20%:50% | 0.073 | 182% |
| Triacetin 20%:Benzyl alcohol | 0.056 | 141% |
| 20%:20%:40% | | |
| PEG 200 30%:20%:50% | 0.049 | 122% |
| Glycerol 30%:20%:50% | 0.061 | 154% |
| Propylene glycol 30%:20%:50% | 0.064 | 160% |
| Labrasol 30%:20%:50% | 0.065 | 162% |
| Capryol 20%:20%:60% | 0.061 | 152% |
| Lauroglycol 20%:20%:60% | 0.065 | 162% |
| Geranyl acetate 20%:20%:60% | 0.053 | 132% |

As explained by Kippen et al., ibid, the above-described PBS diluent system is representative of the solubility of sodium urate/uric acid in the synovial fluid and plasma of a patient.

### Example 4

### Solubility Enhancing Microemulsions

Oleic acid, Labrasol and Transcutol (2-(2-ethoxyethoxy)ethanol) were added to a vial. These components were mixed with stirring at approximately 250 rpm and deionised water was added dropwise. The vials were then shaken for approximately 30 seconds and then stirred for a further 30 minutes to form a solubility enhancing composition of the present invention. The ratios (by weight) of oleic acid:Labrasol:Transcutol:water are given below in Table 5.

The ability of these microemulsions to increase the localised solubility of monosodium urate crystals was then tested using the following method.

A diluent system was prepared to provide a representative imitation of the ionic mixture present in the blood and synovial fluid. An aqueous solution of 0.14 mol/L NaCl was prepared and a 0.01 N phosphate buffer. This solution had a pH of 7.4 and is referred to as “diluent” hereafter.

Approximately 10 mg of MSU crystals was suspended in 10 mL of diluent, and then 1 mL of the microemulsion was added. This was mixed for 16 hours on a roller mixer at room temperature. A sample of the supernatant was taken, filtered through a syringe filter and analysed by HPLC to determine the concentration of uric acid/urate in solution. Results are shown below in Table 4.

TABLE 5
Solubility enhancement of MSU crystals
using microemulsion formulations

| | Concentration | |
|---|---|---|
| Composition (oleic | of Urate | % vs |
| acid:Labrasol:Transcutol:water) | (mg/mL) | control |
| Control (no added microemulsion) | 0.042 | 100% |
| 5:40:20:35 | 0.069 | 164% |
| 5:43.3:21.7:30 | 0.073 | 170% |
| 5:46.7:23.3:25 | 0.076 | 177% |
| 5:50:25:20 | 0.081 | 188% |
| 5:53.3:26.7:15 | 0.080 | 186% |
| 10:36.7:18.3:35 | 0.123 | 286% |
| 10:40:20:30 | 0.115 | 267% |
| 10:43.3:21.7:25 | 0.126 | 293% |
| 10:46.7:23.3:20 | 0.100 | 233% |
| 10:50:25:15 | 0.107 | 249% |
| 10:53.3:26.7:10 | 0.087 | 202% |

As explained by Kippen et al., ibid, the above-described phosphate buffered saline diluent system is representative of the solubility of sodium urate/uric acid in the synovial fluid and plasma of a patient.

### Example 5

### Solubility Enhancement in Franz Cell Experiments

The ability of microemulsions of the present invention to increase the localised solubility of monosodium urate crystals when administered transdermally was then tested using the following method.

Franz cells are used to imitate diffusion through the skin. Franz cell apparatus have two chambers separated by a membrane. In one chamber of the Franz cell, about 10 mg of MSU crystals were suspended in about 10 mL of the diluent solution (as prepared in Example 4). This was stirred at approximately 200 rpm for about 24 hours to achieve an equilibrium saturated suspension. A membrane which was pre-soaked in diluent solution was then clamped as the top of the chamber, in contact with the diluent solution containing the MSU crystal suspension. About 1.5 mL of a microemulsion formulation of the present invention (prepared in Example 4) was then placed on top of the membrane and the cells were covered with lab film. Samples of the supernatant were then taken from the chamber containing the MSU crystal suspension. These were filtered using standard techniques (i.e. a syringe filter) and analysed by HPLC to determine the concentration of uric acid/urate present in the solution. Results of this experiment are shown below in Table 6.

To accurately represent how these formulations would pass through human skin and reach the affected joint, a “Cyclopore®” membrane (Polycarbonate, Whatman® New Jersey, US) was used.

TABLE 6
Solubility enhancement in Franz cell experiments

| | Concentration | |
|---|---|---|
| Franz cell/Microemulsion (oleic | of Urate | % vs |
| acid:Labrasol:Transcutol:water) | (mg/mL) | control |
| Control (no added microemulsion) | 0.042 | 100% |
| 5:40:20:35 | 0.055 | 131% |
| 5:43.3:21.7:30 | 0.055 | 128% |
| 5:46.7:23.3:25 | 0.052 | 121% |
| 5:50:25:20 | 0.054 | 126% |
| 5:53.3:26.7:15 | 0.053 | 123% |
| 10:36.7:18.3:35 | 0.055 | 128% |
| 10:40:20:30 | 0.055 | 128% |
| 10:43.3:21.7:25 | 0.056 | 130% |
| 10:46.7:23.3:20 | 0.058 | 135% |
| 10:50:25:15 | 0.058 | 135% |
| 10:53.3:26.7:10 | 0.056 | 130% |

### Example 6

### Solubility Enhancement in Franz Cell Experiments

The ability of a combination of solubility enhancers of the present invention to increase the localised solubility of monosodium urate crystals when administered transdermally was tested using the following method.

Like Example 5, Franz cells were used to imitate diffusion through the skin. Franz cell apparatus have two chambers separated by a membrane. In one chamber of the Franz cell, about 10 mg of MSU crystals were suspended in about 10 mL of the PBS diluent solution (as prepared in Example 2). A membrane which was pre-soaked in diluent solution was then clamped as the top of the chamber, in contact with the diluent solution containing the MSU crystal suspension. About 0.5 mL of a formulation of the present invention (prepared in Example 2) was then placed on top of the membrane and the cells were covered with lab film. This was stirred at approximately 500 rpm for about 16 hours to achieve an equilibrium saturated suspension. A sample was then taken from the chamber containing the MSU crystal suspension and filtered using standard techniques (i.e. a syringe filter) and analysed by HPLC to determine the concentration of uric acid/urate present in the solution. Results of this experiment are shown below in Table 7.

To accurately represent how these formulations would pass through human skin and reach the affected joint, a “Cycloporee” membrane (Polycarbonate, Whatman® New Jersey, US) was used.

TABLE 7
Solubility enhancement in Franz cell experiments

| Franz cell/Formulation: Composition | Concentration | |
|---|---|---|
| (component 1:Kolliphor ® | of Urate | % vs |
| RH40:Transcutol) | (mg/mL) | control |
| Control (no added formulation) | 0.040 | 100% |
| Triacetin 30%:20%:50% | 0.064 | 159% |
| Benzyl alcohol 30%:20%:50% | 0.077 | 192% |
| Triacetin 20%:Benzyl alcohol | 0.097 | 242% |
| 20%:20%:40% | | |
| PEG 200 30%:20%:50% | 0.055 | 137% |
| Propylene glycol 30%:20%:50% | 0.054 | 134% |
| Labrasol 30%:20%:50% | 0.064 | 160% |

1. A method for treating gout comprising the step of administering an effective amount of a solubility enhancer to a patient in need thereof.

2. The method of claim 1, wherein the solubility enhancer is selected from at least one pharmaceutically acceptable base, at least one solvent, at least one lipid, at least one surfactant, at least one pharmaceutically acceptable acid, at least one cyclodextrin, at least one paraben, or combinations thereof.

3. The method of claim 2, wherein the solubility enhancer is a pharmaceutically acceptable base and is selected from a metal carbonate, a metal hydroxide, a primary amine, a secondary amine, a tertiary amine, an aromatic amine, or a combination thereof.

4. The method of claim 2, wherein the solubility enhancer is a solvent and is selected from an alcohol, a diol, a polyol, an aryl or hereteroaryl alcohol, an arylalkyl or heteroarylalkyl alcohol, an ether, a polyether, a lactam, an amide, an alkyl sulfoxide, a ketone, an aldehyde, a nitrile, an ester, or a combination thereof.

5. The method of claim 2, wherein the solubility enhancer is a lipid and is selected from a C₄-C₂₈ carboxylic acid, a C₁₁-C₂₈ alcohol, a C₁-C₂₈ alkyl C₁-C₂₈ alkanoate, a C₆-C₁₂ monoglyceride, a C₆-C₁₂ diglyceride, a C₆-C₁₂ triglyceride, a C₁-C₂₈ alkyl N,N-disubstituted C₁-C₆ amino C₁-C₂₈ alkanoate, or a combination thereof.

6. The method of claim 2, wherein the solubility enhancer is a surfactant and is selected from a sorbitan ester, an ethoxylated sorbitan ester, a sorbitol ester, an ethoxylated sorbitol ester, a polyoxyethylated castor oil, a polyethoyxlated C₁₁-C₂₈ alcohol, a polyethoxylated C₄-C₂₈ carboxylic acid ester, a polyoxyethylene-polyoxypropylene block copolymer, or a combination thereof.

7. The method of claim 2, wherein the solubility enhancer is a pharmaceutically acceptable acid and is selected from C₁-C₇ carboxylic acid, C₂-C₁₀ dicarboxylic acids, C₁-C₅ alpha hydroxy acids, C₁-C₅ beta hydroxy acid, C₁-C₅ gamma hydroxy acids, sulfonic acids, or a combination thereof.

8. The method of claim 2, wherein the solubility enhancer is a cyclodextrin, and the cyclodextrin is a cyclodextrin having 6-8 glucopyranoside units.

9. The method of claim 2, wherein the solubility enhancer is a paraben and the paraben is a C₁-C₂₀ alkyl paraben.

10. The method of claim 2, wherein the solubility enhancer is selected from sorbitan monooleate, fumaric acid, PEG-8 caprylic caprylo dilglyceride, dimethylformamide, tetraethylene glycol, N-methylpyrrolidone, isopropyl myristate, dimethylacetamide, geranyl acetate, PEG 200, PEG 300, polyoxyethylene (20) sorbitan monooleate, benzyl alcohol, 4-hydroxybenzyl alcohol, PEG-35 castor oil, oleic acid, PEG-40 hydrogenated castor oil, lecithin, benzoic acid, 4-hydroxybenzoic acid, methyl paraben, propyl paraben, salicylic acid or a combination thereof.

11. The method of claim 9, wherein the solubility enhancer is PEG-40 hydrogenated castor oil, 2-(2-ethoxyethoxy)ethanol, oleic acid, benzyl alcohol, 4-hydroxy benzyl alcohol, benzoic acid, 4-hydroxybenzoic acid, methyl paraben, propyl paraben or a combination thereof.

12. The method of claim 1, wherein the method comprises administering the solubility enhancer parenterally to an affected area.

13. The method of claim 1, wherein the method comprises administering the solubility enhancer transdermally to the affected area; preferably wherein the solubility enhancer is administered in combination with a skin permeation enhancer.

14. The method of claim 1, wherein the method comprises administering the solubility enhancer in combination with at least one non-steroidal anti-inflammatory drug, at least one xanthine oxidase inhibitor, colchicine, at least one glucocorticosteroid, or a combination thereof.

15. The method of claim 1, wherein the method comprises administering the solubility enhancer in combination with ultrasound therapy, heat therapy, a change in diet to reduce uric acid levels in the patient, or a combination thereof.

16. The method of claim 1, wherein the solubility enhancer is menthol.
